(19) United States
(12) Patent Application Publication
(10) Pub. No.: US 2023/0097011 A1
(43) Pub. Date: Mar. 30, 2023

(54) A MIXTURE OF MRNA TO ENHANCE THE POTENCY OF DENDRITIC CELLS

(71) Applicant: Vrije Universiteit Brussel, Brussel (BE)

(72) Inventor: Kris THIELEMANS, Antwerpen (BE)

(73) Assignee: Vrije Universiteit Brussel, Brussel (BE)

(21) Appl. No.: 17/911,041

(22) PCT Filed: Mar. 16, 2021
(86) PCT No.: PCT/EP2021/056660

§ 371 Date: Sep. 12, 2022

(30) Foreign Application Priority Data
Mar. 16, 2020 (EP) 20163447.4

(51) Int. Cl. C07K 14/715; C12N 5/0784; C12N 5/10; C07K 14/705

(52) U.S. Cl. CPC C07K 14/7155; C12N 5/0639; C12N 5/10; C07K 14/70596; C07K 14/7156; C07K 14/70575; C12N 2510/00; C12N 2501/231; C12N 2501/65; C12N 2501/2312; A61K 48/00

(57) ABSTRACT

The invention is situated in the field of cancer immunotherapy and more specifically the maturation of antigen-presenting cells in order to enhance their potency to induce an immune response.

### FIELD OF THE INVENTION

The invention is situated in the field of cancer immunotherapy and more specifically the maturation of antigen-presenting cells in order to enhance their potency to induce an immune response.

### BACKGROUND TO THE INVENTION

Dendritic cells (DCs) are one of the most potent antigen-presenting cells of the body and are central in the development of immune responses. A great interest has arisen in loading DCs with tumor-associated antigens expressed by tumors. New DC-based immunotherapy protocols are being conducted through the years in order optimize the outcome of clinical studies with antigen-loaded DCs in cancer patients. However, the desired clinical outcomes showing strong immunological responses are not yet achieved.

Molecules expressed at the surface of DCs (e.g. co-stimulatory molecules) and secreted factors (e.g. cytokines and chemokines) determine the fate of the stimulated effector cells. Among these secreted factors, interleukin-12 (IL-12) is a heterodimeric protein being mainly produced by phagocytes and dendritic cells. It has shown to be a potent activator of innate and adaptive immunity. Furthermore, the secretion of IL-12 has shown to be of importance in the context of cancer-immunotherapy with DCs. By contrast, a number of cytokines have been reported to down-regulate the activation of antitumor immune response. More specifically, interleukin-10 (IL-10) plays a prominent role with this regard. IL-10 produced by dendritic cells (DCs) may influence the DC maturation process and could down-regulate IL-12 production. IL-10 is considered a major marker of tolerogenic DCs. By contrast, it is also shown that IL-10 may represent a potential in tumor immunotherapy in human cancer patients, due to its antitumor immune responses when released locally from transfected tumor cells. Also, the induction of the proliferation and cytotoxic activity of tumor-resident CD8+ T cells by IL-10 is demonstrated as well as the link between IL-10 and an increase of interferon-gamma production in peripheral blood of humans.

These inconclusive results regarding the actual role of IL-10 in regulating immune responses creates uncertainty on best management of IL-10 in the development of DCs having strong immunostimulatory characteristics.

The current invention provides a novel composition of mRNAs, which is capable of modifying the potency of antigen-presenting cells resulting in an enhanced immune response, wherein IL-12 secretion is stimulated, and IL-10 secretion is inhibited.

To our knowledge, the current invention provides a novel approach of developing antigen-loaded antigen-presenting cells which are able to alter IL-12/IL-10 ratios. To achieve this, mRNA encoding for a decoy IL-10 receptor alfa-subunit is introduced within antigen-presenting cells. Furthermore, to our knowledge, no previous links were shown between 11-10 and activation of antigen presenting cells through the decoy IL-10 receptor alfa-subunit as herein provided.

### SUMMARY OF THE INVENTION

The present invention relates to a method for improving the immunostimulatory characteristics of antigen-presenting cells comprising the introduction of mRNA or DNA molecules encoding a decoy IL-10 receptor alfa-subunit in said antigen-presenting cells.

In a following embodiment, in said method mRNA or DNA molecules encoding at least one functional immunostimulatory protein selected from the group comprising: CD70, caTLR4, CD40L and IFN-gamma are further introduced.

In a next embodiment, in said method mRNA or DNA molecules encoding at least one functional immunostimulatory protein selected from the group comprising: caTLR4, CD40L and IFN-gamma are further introduced.

In a further embodiment, in said method mRNA or DNA molecules encoding at least two functional immunostimulatory proteins selected from the group comprising: caTLR4, CD40L and IFN-gamma are further introduced.

In a following embodiment, in said method mRNA or DNA molecules encoding the caTLR4 and IFN-gamma immunostimulatory proteins are further introduced.

In a next embodiment, in said method mRNA or DNA molecules encoding CD40L and/or CD70 immunostimulatory proteins are further introduced.

In yet another embodiment, the present invention provides a method as defined herein wherein the introduction of said mRNA or DNA molecules is obtained via a method selected from the group of electroporation, viral transduction, lipofection or transfection of said antigen-presenting cells.

In a further embodiment, the present invention provides a method as defined herein wherein a contact of IL-10 with said decoy IL-10 receptor alfa-subunit expressing antigen-presenting cells at least results in a stimulation of IL-12 secretion and/or a decrease of IL-10 secretion by said cells. As will become apparent from the examples hereinafter, such increase of IL-12 secretion and/or decrease in IL-10 secretion alters the IL-12/IL10 ratio to an IL-12 excess, that will skew the development of naïve T helper cells (Th) cells toward the memory Th1 phenotype with an enhancement of the antigen-specific cytotoxic activity of the CD8+ cells and NK cells.

In a following embodiment, the present invention relates to a method for preparing an immunotherapy agent comprising the steps of: a) obtaining antigen-presenting cells, b) ex vivo modifying said pool of antigen-presenting cells of step a) according to the method of any of the claims 1-5 and c) ex vivo modifying the pool of antigen-presenting cells from step b) such that they present target-specific antigen derived epitopes.

In a next embodiment, the method of modification used in step c) of said method is selected from the group of electroporation, viral transduction, lipofection or transfection of mRNA or DNA encoding the target-specific antigens.

In another embodiment, in said method the specific immunostimulatory proteins and the target-specific antigens are introduced through a one-step mechanism.

In following embodiment, in said method co-electroporation of the mRNA or DNA encoding a target-specific antigen with the electroporation of the mRNA or DNA molecules encoding the immunostimulatory proteins is used.

In a further embodiment, the present invention provides a method as defined herein, wherein the antigen-presenting cells are selected from the group consisting of Dendritic Cells (DCs) or B-cells isolated from or generated from the blood of a subject, or dendritic cell-lines or B-cell lines.

In a next embodiment, the present invention provides a method as defined herein, wherein the target-specific antigen is selected from the list comprising: tumor, bacterial, viral and fungal antigen.

In a following embodiment, the present invention relates to a composition comprising a combination of mRNA or DNA molecules encoding a decoy IL-10 receptor alfa subunit and mRNA or DNA molecules encoding at least one functional immunostimulatory protein selected from the group comprising: caTLR4, CD40L and IFN-gamma. Characteristic within the compositions according to the invention is that said compositions comprise the combination of polynucleotides (mRNA or DNA) encoding a decoyIL-10 receptor alfa subunit and polynucleotides (mRNA or DNA) encoding a least one functional immunostimulatory protein selected from the group comprising: caTLR4, CD40L and IFN-gamma. In a particular embodiment the compositions comprise the combination of mRNA encoding a decoyIL-10 receptor alfa subunit and mRNA encoding a least one functional immunostimulatory protein selected from the group comprising: caTLR4, CD40L and IFN-gamma.

In a next embodiment, the present invention relates to a composition comprising a combination of mRNA or DNA molecules encoding a decoy IL-10 receptor alfa subunit and mRNA or DNA molecules encoding at least two functional immunostimulatory proteins selected from the group comprising: caTLR4, CD40L and IFN-gamma. Characteristic within the compositions according to the invention is that said compositions comprise the combination of polynucleotides (mRNA or DNA) encoding a decoyIL-10 receptor alfa subunit and polynucleotides (mRNA or DNA) encoding a least two functional immunostimulatory protein selected from the group comprising: caTLR4, CD40L and IFN-gamma. In a particular embodiment the compositions comprise the combination of mRNA encoding a decoyIL-10 receptor alfa subunit and mRNA encoding a least two functional immunostimulatory protein selected from the group comprising: caTLR4, CD40L and IFN-gamma.

In yet another embodiment, the present invention relates to a composition comprising a combination of mRNA or DNA molecules encoding a decoy IL-10 receptor alfa subunit, caTLR4, CD40L and IFN-gamma immunostimulatory proteins. In a particular embodiment the compositions comprising mRNA molecules encoding a decoy IL-10 receptor alfa subunit, caTLR4, CD40L and IFN-gamma immunostimulatory proteins.

In a next embodiment, the present invention relates to a composition comprising a combination of mRNA or DNA molecules encoding a decoy IL-10 receptor alfa subunit, caTLR4 and IFN-gamma immunostimulatory proteins.

In a following embodiment, said composition further comprises mRNA or DNA molecules encoding CD40L and/or CD70 immunostimulatory proteins.

In a further embodiment, the composition as defined herein further comprises pharmaceutically acceptable adjuvant(s).

In a next embodiment, the present invention relates to a composition as defined herein for use in the treatment of tumor presence, cancer, IL-10 related conditions, bacterial, viral or fungal infection, HIV infection or hepatitis infection.

In a following embodiment, the present invention relates to a use of a composition as defined herein as an immunostimulatory agent capable of at least potentiating an immune response in a patient in need thereof.

In yet another embodiment, said patient is suffering from a disease or disorder selected from the group of: tumor presence, cancer, IL-10 related conditions, bacterial, viral or fungal infection, HIV infection or hepatitis infection.

The invention can also be summarized by the following numbered embodiments;
1. A method for improving the immunostimulatory characteristics of antigen-presenting cells comprising the introduction of mRNA or DNA molecules encoding a decoy IL-10 receptor alfa-subunit in said antigen-presenting cells.
2. The method of embodiment 1, wherein mRNA or DNA molecules encoding at least one functional immunostimulatory protein selected from the group comprising: CD70, caTLR4, CD40L and IFN-gamma are further introduced
3. The method of embodiment 1, wherein mRNA or DNA molecules encoding at least one functional immunostimulatory protein selected from the group comprising: caTLR4, CD40L and IFN-gamma are further introduced.
4. The method of embodiment 1, wherein mRNA or DNA molecules encoding at least two functional immunostimulatory proteins selected from the group comprising: caTLR4, CD40L and IFN-gamma are further introduced.
5. The method of embodiment 1, wherein mRNA or DNA molecules encoding the caTLR4 and IFN-gamma immunostimulatory proteins are further introduced.
6. The method of embodiment 5, wherein mRNA or DNA molecules encoding CD40L and/or CD70 immunostimulatory proteins are further introduced.
7. The method of any of the embodiments 1-5, wherein the introduction of said mRNA or DNA molecules is obtained via a method selected from the group of electroporation, viral transduction, lipofection or transfection of said antigen-presenting cells.
8. The method of any of the embodiments 1-6, wherein a contact of IL-10 with said antigen-presenting cells at least results in a stimulation of IL-12 secretion and/or a decrease of IL-10 secretion.
9. A method for preparing an immunotherapy agent comprising the steps of:
   a) obtaining antigen-presenting cells;
   b) ex vivo modifying said pool of antigen-presenting cells of step a) according to the method of any of the claims 1-5;
   c) ex vivo modifying the pool of antigen-presenting cells from step b) such that they present target-specific antigen derived epitopes.
10. The method of embodiment 9, wherein the method of modification used in step c) is selected from the group of electroporation, viral transduction, lipofection or transfection of mRNA or DNA encoding the target-specific antigens.
11. The method of any of the embodiments 9-10, wherein the specific immunostimulatory proteins and the target-specific antigens are introduced through a one-step mechanism.
12. The method of embodiment 8, wherein co-electroporation of the mRNA or DNA encoding a target-specific antigen with the electroporation of the mRNA or DNA molecules encoding the immunostimulatory proteins, is used.
13. The method of any of the embodiments 1-12, wherein the antigen-presenting cells are selected from the group consisting of Dendritic Cells (DCs) or B-cells isolated from or generated from the blood of a subject, or dendritic cell-lines or B-cell lines.
14. The method of any of the embodiments 1-13, wherein the target-specific antigen is selected from the list comprising: tumor, bacterial, viral and fungal antigen.
15. A composition comprising a combination of polynucleotides (i.e. mRNA or DNA) molecules encoding a decoy IL-10 receptor alfa subunit and polynucleotides (mRNA or DNA) molecules encoding at least one functional immunostimulatory protein selected from the group comprising: CD70, caTLR4, CD40L and IFN-gamma.
16. A composition comprising a combination of polynucleotides (mRNA or DNA) molecules encoding a decoy IL-10 receptor alfa subunit and mRNA or DNA molecules encoding at least two functional immunostimulatory proteins selected from the group comprising: CD70, caTLR4, CD40L and IFN-gamma.
17. A composition comprising a combination of polynucleotides (mRNA or DNA) molecules encoding a decoy IL-10 receptor alfa subunit, caTLR4, CD40L and IFN-gamma immunostimulatory proteins.
18. A composition comprising a combination of polynucleotides (mRNA or DNA) molecules encoding a decoy IL-10 receptor alfa subunit, caTLR4 and IFN-gamma immunostimulatory proteins.
19. The composition of embodiment 18, further comprising polynucleotides (mRNA or DNA) molecules encoding CD40L and/or CD70 immunostimulatory proteins.
20. The composition of any of the embodiments 15-18, further comprising pharmaceutically acceptable adjuvant(s).
21. A composition according to any of the embodiments 15-18 for use in the treatment of tumor presence, cancer, IL-10 related conditions, bacterial, viral or fungal infection, HIV infection or hepatitis infection.
22. Use of a composition according to any of the embodiments 15-19 as an immunostimulatory agent capable of at least potentiating an immune response in a patient in need thereof.
23. The use of a composition according to embodiment 20, wherein the patient is suffering from a disease or disorder selected from the group of: tumor presence, cancer, IL-10 related conditions, bacterial, viral or fungal infection, HIV infection or hepatitis infection.

### BRIEF DESCRIPTION OF THE DRAWINGS

FIG. 1 shows the results of an in vitro stimulation of MelanA-specific T cells with TriMix- and TetraMix modified moDC, according to an embodiment of the present invention.

FIG. 2 shows the results of a comparison of IL-12/IL-10 ratios for TriMix- and TetraMix modified moDCs.

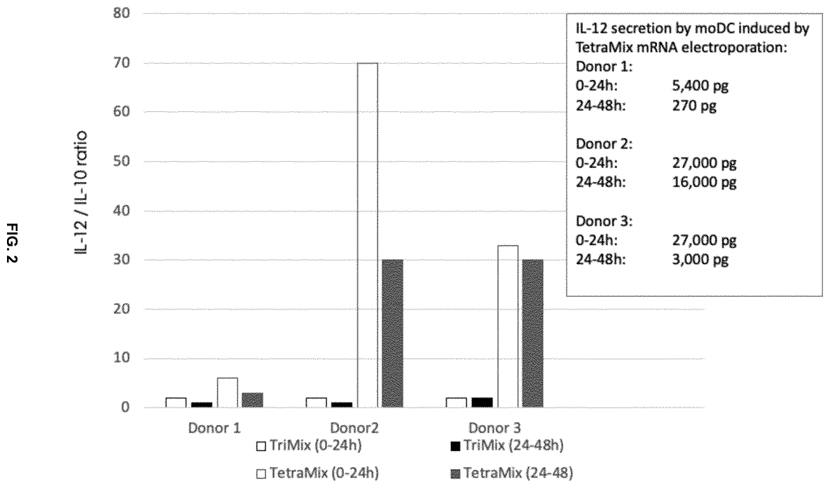

### DETAILED DESCRIPTION OF THE INVENTION

The present invention will be described with respect to particular embodiments and with reference to certain drawings, but the invention is not limited thereto. The drawings, as further described, are only schematic and non-limiting.

Furthermore, the terms first, second, further and the like in the description and in the claims are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a composition comprising A and B” should not be limited to products consisting only of elements A and B. It means that, with respect to the present invention, the relevant elements of the composition are A and B and that further components such as C may be present.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some, but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The term “target” used throughout the description is not limited to the specific examples that may be described herein. Any infectious agent such as a virus, a bacterium or a fungus may be targeted. In addition any tumor or cancer cell may be targeted.

The term “target-specific antigen” used throughout the description is not limited to the specific examples that may be described herein. It will be clear to the skilled person that the invention is related to the induction of immunostimulation in antigen presenting cells, regardless of the target-specific antigen that is presented. The antigen that is to be presented will depend on the type of target to which one intends to elicit an immune response in a subject. Typical examples of target-specific antigens are expressed or secreted markers that are specific to tumor, bacterial and fungal cells or to specific viral proteins or viral structures.

The term “antigen presenting cell” used throughout the description includes all antigen presenting cells. Specific non limiting examples are dendritic cells, dendritic cell-lines, b-cells, or B-cell-lines. The dendritic cells or B-cells can be isolated or generated from the blood of a patient or healthy subject. The patient or subject can have been the subject of prior vaccination or not.

The terms “cancer” and/or “tumor” used throughout the description are not intended to be limited to the types of cancer or tumors that may have been exemplified. The term therefore encompasses all proliferative disorders such as neoplasma, dysplasia, premalignant or precancerous lesions, abnormal cell growths, benign tumors, malignant tumors, cancer or metastasis, wherein the cancer is selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma.

The term “infectious disease” or “infection” used throughout the description is not intended to be limited to the types of infections that may have been exemplified herein. The term therefore encompasses all infectious agents to which vaccination would be beneficial to the subject. Non-limiting examples are the following virus-caused infections or disorders: Acquired Immunodeficiency Syndrome-Adenoviridae Infections-Alphavirus Infections-Arbovirus Infections-Bell Palsy-Borna Disease-Bunyaviridae Infections-Caliciviridae Infections-Chickenpox-Common Cold-Condyloma Acuminata-Coronaviridae Infections-Coxsackievirus Infections-Cytomegalovirus Infections-Dengue-DNA Virus Infections-Contagious Ecthyma, -Encephalitis-Encephalitis, Arbovirus-Encephalitis, Herpes Simplex-Epstein-Barr Virus Infections-Erythema Infectiosum-Exanthema Subitum-Fatigue Syndrome, Chronic-Hantavirus Infections-Hemorrhagic Fevers, Viral-Hepatitis, Viral, Human-Herpes Labialis-Herpes Simplex-Herpes Zoster-Herpes Zoster Oticus-Herpesviridae Infections-HIV Infections-Infectious Mononucleosis-Influenza in Birds-Influenza, Human-Lassa Fever -Measles-Meningitis, Viral-Molluscum Contagiosum-Monkeypox-Mumps-Myelitis-Papillomavirus Infections-Paramyxoviridae Infections-Phlebotomus Fever-Poliomyelitis-Polyomavirus Infections-Postpoliomyelitis Syndrome-Rabies-Respiratory Syncytial Virus Infections-Rift Valley Fever-RNA Virus Infections-Rubella-Severe Acute Respiratory Syndrome-Slow Virus Diseases-Smallpox-Subacute Sclerosing Panencephalitis-Tick-Borne Diseases-Tumor Virus Infections-Warts-West Nile Fever-Virus Diseases-Yellow Fever-Zoonoses -Etc. Specific antigens for viruses can be HIV-gag,-tat, -rev or -nef, or Hepatitis C-antigens.

Further non-limiting examples are the following bacteria- or fungus-caused infections or disorders: Abscess-Actinomycosis-Anaplasmosis-Anthrax-Arthritis, Reactive-Aspergillosis -Bacteremia-Bacterial Infections and Mycoses-Bartonella Infections-Botulism-Brain Abscess-Brucellosis-Burkholderia Infections-Campylobacter Infections-Candidiasis-Candidiasis, Vulvovaginal-Cat-Scratch Disease-Cellulitis-Central Nervous System Infections -Chancroid-Chlamydia Infections-Chlamydiaceae Infections-Cholera-Clostridium Infections-Coccidioidomycosis -Corneal Ulcer-Cross Infection-Cryptococcosis-Dermatomycoses-Diphtheria-Ehrlichiosis -Empyema, Pleural-Endocarditis, Bacterial-Endophthalmitis-Enterocolitis, Pseudomembranous-Erysipelas-Escherichia coli Infections-Fasciitis, Necrotizing-Fournier Gangrene-Furunculosis-Fusobacterium Infections-Gas Gangrene-Gonorrhea-Gram-Negative Bacterial Infections-Gram-Positive Bacterial Infections-Granuloma Inguinale-Hidradenitis Suppurativa-Histoplasmosis-Hordeolum-Impetigo-Klebsiella Infections-Legionellosis-Leprosy-Leptospirosis-Listeria Infections-Ludwig's Angina-Lung Abscess-Lyme Disease- Lymphogranuloma Venereum-Maduromycosis-Melioidosis-Meningitis, Bacterial-Mycobacterium Infections-Mycoplasma Infections-Mycoses-Nocardia Infections-Onychomycosis-Osteomyelitis-Paronychia-Pelvic Inflammatory Disease-Plague-Pneumococcal Infections-Pseudomonas Infections-Psittacosis-Puerperal Infection-Q Fever-Rat-Bite Fever-Relapsing Fever-Respiratory Tract Infections-Retropharyngeal Abscess-Rheumatic Fever-Rhinoscleroma-Rickettsia Infections-Rocky Mountain Spotted Fever-Salmonella Infections-Scarlet Fever-Scrub Typhus-Sepsis-Sexually Transmitted Diseases, Bacterial-Sexually Transmitted Diseases, Bacterial-Shock, Septic-Skin Diseases, Bacterial-Skin Diseases, Infectious-Staphylococcal Infections-Streptococcal Infections-Syphilis-Syphilis, Congenital-Tetanus-Tick-Borne Diseases-Tinea-Tinea Versicolor-Trachoma-Tuberculosis-Tuberculosis, Spinal-Tularemia-Typhoid Fever-Typhus, Epidemic Louse-Borne-Urinary Tract Infections-Whipple Disease-Whooping Cough-Vibrio Infections-Yaws-Yersinia Infections-Zoonoses or Zygomycosis.

As already detailed herein before, in a first aspect, the present invention provides a method for improving the immunostimulatory characteristics of antigen-presenting cells comprising the introduction of mRNA or DNA molecules encoding a decoy IL-10 receptor alfa-subunit in said antigen-presenting cells.

As used herein and unless otherwise specified, the term “decoy IL-10 receptor alfa-subunit” is to be understood as variant of the IL-10 receptor alfa-subunit lacking the intracellular domain and the associated JAK1.

As used herein and unless otherwise specified, the term “IL-10” is to be understood as a cytokine with multiple, pleiotropic effects in immunoregulation and inflammation. IL-10 plays a crucial role in maintaining a balance between effective resistance against pathogens and severe systemic inflammation. IL-10 is encoded in humans by the IL10 gene.

As used herein and unless otherwise specified, the term “IL-12” is to be understood as a cytokine being mainly produced by phagocytes and DCs in response to antigenic stimulation. IL-12 primarily acts on natural killer cells and T cells and induces T cells to acquire a type 1 differentiation profile characterized by an increased production of interferon-gamma (IFN-gamma). IL-12 is a potent activator of innate and adaptive immunity.

For example, when induced in DCs, IL-10 is a potent inhibitor of DC functions of which the inhibition of IL-12 production is an important example in the present context. This IL-12 (may also be called “IL-12p70”) inhibition is effectuated by blocking the transcription of both of the IL-12 encoding genes, being p35 and p40, through induction of the synthesis of an as-yet unidentified protein.

In some embodiments, the method may be used for in vivo applications.

In a following embodiment, in said method of introduction of polynucleotide (mRNA or DNA) molecules encoding a decoy IL-10 receptor alfa-subunit in said antigen-presenting cells, and polynucleotide (mRNA or DNA) molecules encoding at least one functional immunostimulatory protein selected from the group comprising: CD70, caTLR4, CD40L and IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70 are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding caTLR4 are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD40L are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70 and caTLR4 are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70 and CD40L are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70 and IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding caTLR4 and CD40L are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding caTLR4 and IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD40L and IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70, caTLR4 and CD40L are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70, caTLR4 and IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding CD70, CD40L and IFN-gamma are further introduced.

In some embodiments, in said method of introduction of polynucleotide (mRNA or DNA) molecules encoding a decoy IL-10 receptor alfa-subunit in said antigen-presenting cells, polynucleotide (mRNA or DNA) molecules encoding caTLR4, CD40L and IFN-gamma are further introduced.

In some embodiments, the further introduction comprises a co-electroporation of said antigen-presenting cells with at least one of said immunostimulatory proteins.

As used herein and unless otherwise specified, the term “CD40 ligand (CD40L)” is to be understood as a potent DC activation protein which binds to the C40 protein on antigen-presenting cells. It may also be referred to as “CD154”. The expression of CD40L on DCs may induce their activation by ligation to endogenous CD40 receptor. CD40-CD40L interactions mediate one of the most potent DC activating signals. Commonly, CD40 ligation on DCs is provided by activated CD4+ T cells. This process which may be simulated by engineering DCs to express CD40L, may lead to an upregulation of co-stimulatory molecules and enhanced production of cytokines and/or chemokines. It is shown that CD40 ligation increases the magnitude of CD4+ and CD8+ T-cell expansion. Especially for the induction of memory CD8+ T cells, CD40 ligation is important.

As used herein and unless otherwise specified, the term “constitutively active Toll-like receptor 4 (caTLR4)” is to be understood as a constitutively active form of TLR4 which is able to mimic the effect of lipopolysaccharide (LPS) binding on DCs once expressed by DCs. CaTLR4 receptor expression on DCs induces their activation. The binding of pathogen-associated molecular patterns to toll-like receptors (TLRs) provides important signals for DC maturation. Ligation of TLRs induces similar effects as CD40 ligation on DCs, namely, upregulation of co-stimulatory molecules and enhanced cytokine/chemokine secretion. Among the TLR ligands, LPS, which binds to TLR4, is an attractive candidate because LPS-matured DCs have been shown to acquire an enhanced ability to stimulate specific T cells.

As used herein and unless otherwise specified, the term “Interferon gamma (IFN-gamma)” is to be understood as a cytokine of the type II class interferons which fulfils an important role as activator of macrophages, inducer of class II major histocompatibility complex (MHC) molecule expression and effectuating immunostimulatory and immunomodulatory effects. Since the transcriptional activation of IL-12p70 is dependent on two signals, one initiated by CD40 or TLR and the other initiated by IFN-gamma, IFN-gamma signals are effectuating primarily IL-12p35 transcriptional activation.

In a next embodiment, in said method of introduction of mRNA or DNA; in particular mRNA molecules encoding a decoy IL-10 receptor alfa-subunit in said antigen-presenting cells, mRNA or DNA; in particular mRNA molecules encoding at least one functional immunostimulatory protein selected from the group comprising: caTLR4, CD40L and IFN-gamma are further introduced.

In yet another embodiment, in said method of introduction of mRNA or DNA; in particular mRNA molecules encoding a decoy IL-10 receptor alfa-subunit in said antigen-presenting cells, mRNA or DNA; in particular mRNA molecules encoding at least two functional immunostimulatory proteins selected from the group comprising: caTLR4, CD40L and IFN-gamma are further introduced.

In a following embodiment, in said method of introduction of mRNA or DNA; in particular mRNA molecules encoding a decoy IL-10 receptor alfa-subunit in said antigen-presenting cells, mRNA or DNA; in particular mRNA molecules encoding the caTLR4 and IFN-gamma immunostimulatory proteins are further introduced.

The production of bioactive IL-12p70 depends among others on the transcriptional regulation of genes encoding both IL-12p35 and IL-12p40 subunits. Also, the transcriptional activation of IL-12p70 depends on two signals, the first one initiated by the immunostimulatory proteins CD40 or TLR and the second one by IFN-gamma. IL-12 is a T cell-stimulating factor, supporting the differentiation of naïve T cells into Th1 cells and mediating the enhancement of cytotoxic activity of CD8+ T cells and NK cells.

In a next embodiment, in said method of introduction of mRNA or DNA; in particular mRNA molecules encoding a decoy IL-10 receptor alfa-subunit in said antigen-presenting cells, the caTLR4 and IFN-gamma immunostimulatory proteins; mRNA or DNA; in particular mRNA molecules encoding CD40L and/or CD70 immunostimulatory proteins are further introduced.

In yet another embodiment, the present invention provides a method as defined herein wherein the introduction of said mRNA or DNA molecules; in particular of mRNA molecules is obtained via a method selected from the group of electroporation, viral transduction, lipofection or transfection of said antigen-presenting cells.

In preferred embodiments, said introduction is obtained via electroporation.

In a further embodiment, the present invention provides a method as defined herein wherein a contact of IL-10 with said decoy IL-10 receptor alfa-subunit expressing antigen-presenting cells at least results in a stimulation of IL-12 secretion and/or a decrease of IL-10 secretion by said cells.

The introduction of mRNA or DNA; in particular of mRNA molecules encoding the decoy IL-10 receptor alfa subunit in said antigen-presenting cells (e.g. DCs) and the expression thereof in said antigen-presenting cells may result in the binding of IL-10 with the decoy IL-10 receptor alfa subunit, in order to inhibit the formation of a fully biologically active IL-10-IL-10-receptor alfa-IL-10 receptor beta complex. The decoy IL-10 receptor alfa-subunit complex will compete with the endogenous IL-10 receptor alfa chain for IL-10 binding. This ultimately results in less inhibition of IL-12 subunits (e.g. IL-12p35 and/or IL-12p40) transcription and, therefore, a higher IL-12 secretion in DCs.

In some embodiments, said contact may alter the ratio of the IL-12/IL-10.

In some embodiments, said contact may effectuate a higher secretion of IL-12 resulting in a higher IL-12/IL-10 ratio.

In a following embodiment, the present invention relates to a method for preparing an immunotherapy agent comprising the steps of: a) obtaining antigen-presenting cells, b) ex vivo modifying said pool of antigen-presenting cells of step a) according to the methods of the invention, such as in any of the claims 1-5 and c) ex vivo modifying the pool of antigen-presenting cells from step b) such that they present target-specific antigen derived epitopes.

In a next embodiment, the method of modification used in step c) of said method is selected from the group of electroporation, viral transduction, lipofection or transfection of mRNA or DNA encoding the target-specific antigens.

In another embodiment, in said method the specific immunostimulatory proteins and the target-specific antigens are introduced through a one-step mechanism.

In following embodiment, in said method co-electroporation of the mRNA or DNA; in particular of the mRNA encoding a target-specific antigen with the electroporation of the mRNA or DNA; in particular of mRNA molecules encoding the immunostimulatory proteins is used.

In a further embodiment, the present invention provides a method as defined herein, wherein the antigen-presenting cells are selected from the group consisting of Dendritic Cells (DCs) or B-cells isolated from or generated from the blood of a subject, or dendritic cell-lines or B-cell lines.

In a next embodiment, the present invention provides a method as defined herein, wherein the target-specific antigen is selected from the list comprising: tumor, bacterial, viral and fungal antigen.

In a following embodiment, the present invention relates to a composition comprising a combination of polynucleotide (mRNA or DNA) molecules encoding a decoy IL-10 receptor alfa subunit and polynucleotide (mRNA or DNA) molecules encoding at least one functional immunostimulatory protein selected from the group comprising: caTLR4, CD40L and IFN-gamma.

In a next embodiment, the present invention relates to a composition comprising a combination of mRNA or DNA; in particular mRNA molecules encoding a decoy IL-10 receptor alfa subunit and mRNA or DNA; in particular mRNA molecules encoding at least two functional immunostimulatory proteins selected from the group comprising: caTLR4, CD40L and IFN-gamma.

In some embodiments, the composition may be used for in vivo applications.

In a next embodiment, the present invention relates to a composition comprising a combination of mRNA or DNA; in particular mRNA molecules encoding a decoy IL-10 receptor alfa subunit, caTLR4, CD40L and IFN-gamma immunostimulatory proteins.

In yet another embodiment, the present invention relates to a composition comprising a combination of mRNA or DNA; in particular mRNA molecules encoding a decoy IL-10 receptor alfa subunit, caTLR4 and IFN-gamma immunostimulatory proteins.

In a following embodiment, said composition further comprises mRNA or DNA; in particular mRNA molecules encoding CD40L and/or CD70 immunostimulatory proteins.

In a further embodiment, the composition as defined herein further comprises pharmaceutically acceptable adjuvant(s).

In a next embodiment, the present invention relates to a composition as defined herein for use in the treatment of tumor presence, cancer, IL-10 related conditions, bacterial, viral or fungal infection, HIV infection or hepatitis infection.

In a following embodiment, the present invention relates to a use of a composition as defined herein as an immunostimulatory agent capable of at least potentiating an immune response in a patient in need thereof.

In some embodiments, said composition may be used in the preparation of TetraMix-DC vaccines, which may be used for at least one of said treatments.

In some embodiments, said TetraMix-DC vaccine may be used as an anti-cancer vaccine.

Unless provided otherwise, the term “TetraMix” should be understood as a mixture of mRNA molecules encoding a decoy IL-10 receptor alfa subunit, caTLR4, CD40L and IFN-gamma immunostimulatory proteins.

Unless provided otherwise, the term “TetraMix DCs” or “TetraMix antigen presenting cells” stands for respectively dendritic cells or antigen presenting cells that have been modified to express the TetraMix mixture of mRNA molecules encoding a decoy IL-10 receptor alfa subunit, caTLR4, CD40L and IFN-gamma immunostimulatory proteins.

In some embodiments, said immune response may be a type 1 T helper cell (TH1)/T cytotoxic cell type 1 (TC1) immune response.

In yet another embodiment, said patient is suffering from a disease or disorder selected from the group of: tumor presence, cancer, IL-10 related conditions, bacterial, viral or fungal infection, HIV infection or hepatitis infection.

### EXAMPLES

The invention is illustrated by the following non-limiting examples

### Example 1: Comparison of IL-12 and IL-10 Secretion by DCs Electroporated with TriMix mRNA and TetraMix mRNA

The test results of the enhanced IL-12 and suppressed IL-10 secretion upon electroporation of DCs with TetraMix mRNA compared to TriMix mRNA electroporated moDCs are summarized in the table below. Unless provided otherwise, the term “TriMix” stands for the specific combination of CD40L, CD70 and caTLR4.

The values are given in pg/ml per 10⁶DCs during the first 24 h after electroporation of the DCs.

| | Interleukin 12 | | | Interleukin 10 | | |
|---|---|---|---|---|---|---|
| Donor | TriMix | TetraMix | x increase | TriMix | TetraMix | x decrease |
| DWM | 203 | 20.429 | 100 | 11.655 | 3.726 | 3 |
| DMV | 1.652 | 237.276 | 144 | 11.008 | 4.184 | 2.6 |
| DTP | 395 | 364 | 0.9 | 18.255 | 863 | 21 |
| DJM | 1.519 | 121.120 | 80 | 215 | 218 | 0.9 |
| DJS | 257 | 11.482 | 47 | 1.920 | 413 | 4.6 |
| DPS | 40 | 762 | 19 | 926 | 553 | 1.7 |
| DLS | 14 | 5.399 | 385 | 1.549 | 860 | 1.8 |
| DHD | 9.144 | 27.674 | 3 | | | |
| DCJ | 268 | 27.419 | 102 | | | |
| Mean | | | 85 × more | | | 5 × less |

### Example 2: In Vitro Stimulation of MelanA-Specific T Cells with TriMix- and TetraMix Modified moDC

The T cell stimulatory capacity of TetraMix mRNA modified DCs are measured by executing an in vitro stimulation assay. Monocyte derived HLA-A2+ DC were electroporated with MelanA mRNA and TriMix or TetraMix mRNA. These cells were then used to stimulate CD8+ T cells in vitro. After 3 rounds of stimulation, the MelanA specific T cells were detected by staining with pMHC-multimers. TetraMixDC-MelanA induced a higher number of MelanA-specific T cells. The values are expressed as % specific T cells among the CD8+ T cells.

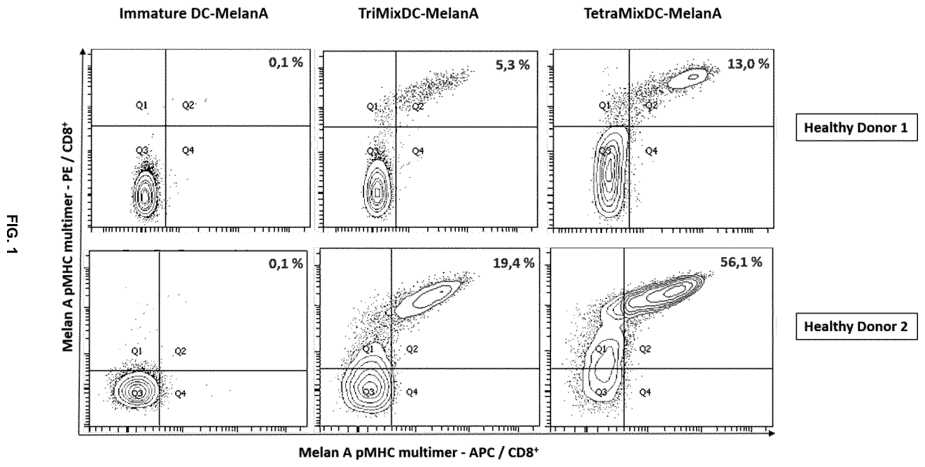

See also FIG. 1.

### Example 3: Comparison of IL-12/IL-10 Ratios for TriMix- and TetraMix Modified moDCs

SeefFgure 2. Shows the results of a comparison of IL-12/IL-10 ratios for moDCs electroporated with the TriMix mRNA mix on the one hand and the TetraMix mRNA mix on the other hand. Differing in the absence, respectively presence of an mRNA molecules encoding a decoy IL-10 receptor alfa subunit (Seq ID. No. 4), this, as well as Examples 1 and 2, show the effect of the decoy on the IL-12/IL-10 ratio and corresponding differentiation of the DCs into Th1-cells with an enhancement of the antigen-specific cytotoxic activity of the CD8+ cells and NK cells.

The aim of the electroporation of moDCs with the mRNA mixes is to reprogram the cells to increase the secretion of the immuno-stimulatory cytokine IL-12 and to inhibit the autocrine effect of the immune-suppressive cytokine IL-10. A higher amount of IL-12 and a lower amount of IL-10 will result in a higher IL-12/IL-10 ratio. The amount of these cytokines secreted in the culture medium, is determined by ELISA. The amount of the cytokines secreted during the first 24 hrs after the electroporation (0-24 h) and during the following 24 hrs (24-48 h) is determined.

The results shown in the figure, illustrate the effect of the modification of moDCs with TetraMix- and TriMix-mRNA. The IL-12/IL-10 ratio is much higher when the DCs are electroporate with TetraMix mRNA, both during the first 14 h as well during the next 24 h of the culture of the cells.

SEQUENCE LIST
TLR4ca mRNA:
(SEQ ID No: 1)
GGCCGGCGGGUUUCUGACAUCCGGCGGGUUUCUGACAUCCGGCGGGUUUC
UGACAUCCGGCGGGUUUCUGACAUCCGGCGGGUUUCUGACAUCCGGCGGG
UUUCUGACAUCCGGCGGGUUUCUGACAUCCGGCGGGUUUCUGACAUCCGG
CGGGUUUCUGACAUCCGGCGGGUUUCUGACAUUCACAACCAGGCCUCCAC
AACCAUGGCUGCCCCUGGCGCUAGAAGGCCUCUUCUCCUUCUGCUGCUGG
CCGGACUGGCUCAUGGCGCCUCUGCCCUGUUUGAGGACCCUGUGCUGAGC
CUGAACAUCACCUGUCAGAUGAACAAGACCAUCAUCGGCGUGUCCGUGCU
GAGCGUGCUGGUGGUGUCUGUGGUGGCUGUGCUGGUGUACAAGUUCUACU
UCCACCUGAUGCUGCUGGCUGGCUGCAUUAAGUACGGCAGGGGCGAGAAC
AUCUACGACGCCUUCGUGAUCUACAGCAGCCAGGACGAGGACUGGGUGCG
CAACGAGCUCGUGAAGAACCUGGAAGAGGGCGUGCCCCCAUUCCAGCUGU
GCCUGCACUACCGGGACUUCAUCCCCGGCGUGGCCAUUGCCGCCAACAUC
AUCCACGAGGGCUUCCACAAGAGCCGGAAAGUGAUCGUGGUGGUGUCCCA
GCACUUCAUCCAGAGCCGGUGGUGCAUCUUCGAGUACGAGAUCGCCCAGA
CCUGGCAGUUCCUGAGCAGCAGAGCCGGCAUCAUCUUCAUCGUGCUGCAG
AAGGUGGAAAAGACCCUGCUGAGACAACAGGUGGAACUGUACCGGCUGCU
GAGCAGAAACACCUACCUGGAAUGGGAGGACUCCGUGCUGGGCAGACACA
UCUUCUGGCGGAGACUGCGGAAGGCCCUGCUGGAUGGCAAGAGCUGGAAU
CCCGAGGGCACAGUGGGCACCGGCUGCAAUUGGCAGGAAGCCACCAGCAU
CUGAUAACUCGAGUGUUUUGGCUGGGUUUUUCCUUGUUCGCACCGGACAC
CUCCAGUGACCAGACGGCAAGGUUUUUAUCCCAGUGUAUAUUGUCGACAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAA
CD40L mRNA
(SEQ ID No: 2)
GGCCGGCGGGUUUCUGACAUCCGGCGGGUUUCUGACAUCCGGCGGGUUUC
UGACAUCCGGCGGGUUUCUGACAUCCGGCGGGUUUCUGACAUCCGGCGGG
UUUCUGACAUCCGGCGGGUUUCUGACAUCCGGCGGGUUUCUGACAUCCGG
CGGGUUUCUGACAUCCGGCGGGUUUCUGACAUUCACAACCAGGCCUCCAC
AACCAUGGUCGAGACAUACAACCAGACCAGCCCCAGAAGCGCCGCCACAG
GCCUGCCUAUCAGCAUGAAGAUCUUUAUGUAUCUGCUGACCGUGUUCCUG
AUCACCCAGAUGAUCGGCAGCGCCCUGUUCGCCGUGUAUCUGCACAGACG
GCUGGACAAGAUCGAGGACGAGCGGAAUCUGCACGAGGACUUCGUGUUCA
UGAAGACCAUCCAGCGGUGCAACACCGGCGAGAGAAGCCUGAGCCUGCUG
AACUGCGAGGAAAUCAAGAGCCAGUUCGAGGGCUUCGUGAAGGACAUCAU
GCUGAACAAAGAGGAAACUAAGAAAGAAAACAGCUUCGAGAUGCAGAAGG
GCGACCAGAACCCCCAGAUUGCCGCCCACGUGAUCAGCGAGGCCAGCAGC
AAGACCACCUCCGUGCUGCAGUGGGCCGAGAAGGGCUACUACACCAUGAG
CAACAACCUCGUGACCCUGGAAAACGGCAAGCAGCUGACAGUGAAGCGGC
AGGGCCUGUACUACAUCUACGCCCAAGUGACCUUCUGCAGCAACAGAGAG
GCCAGCUCCCAGGCCCCCUUUAUCGCCAGCCUGUGCCUGAAGUCCCCCGG
CAGAUUCGAGCGGAUCCUGCUGAGAGCCGCCAACACACACAGCAGCGCCA
AGCCUUGUGGCCAGCAGUCUAUCCACCUGGGCGGCGUGUUCGAACUGCAG
CCUGGCGCCUCCGUGUUCGUGAACGUGACCGAUCCUAGCCAGGUGUCCCA
CGGCACCGGCUUCACAAGCUUCGGACUGCUGAAGCUGUGAUGACUCGAGU
GUUUUGGCUGGGUUUUUCCUUGUUCGCACCGGACACCUCCAGUGACCAGA
CGGCAAGGUUUUUAUCCCAGUGUAUAUUGUCGACAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAA
IFN-gamma mRNA
(SEQ ID No: 3)
GGCCGGCGGGUUUCUGACAUCCGGCGGGUUUCUGACAUCCGGCGGGUUUC
UGACAUCCGGCGGGUUUCUGACAUCCGGCGGGUUUCUGACAUCCGGCGGG
UUUCUGACAUCCGGCGGGUUUCUGACAUCCGGCGGGUUUCUGACAUCCGG
CGGGUUUCUGACAUCCGGCGGGUUUCUGACAUUCACAACCAGGCCUCCAC
AACAUGAAAUAUACAAGUUAUAUCUUGGCUUUUCAGCUCUGCAUCGUUUU
GGGUUCUCUUGGCUGUUACUGCCAGGACCCAUAUGUAAAAGAAGCAGAAA
ACCUUAAGAAAUAUUUUAAUGCAGGUCAUUCAGAUGUAGCGGAUAAUGGA
ACUCUUUUCUUAGGCAUUUUGAAGAAUUGGAAAGAGGAGAGUGACAGAAA
AAUAAUGCAGAGCCAAAUUGUCUCCUUUUACUUCAAACUUUUUAAAAACU
UUAAAGAUGACCAGAGCAUCCAAAAGAGUGUGGAGACCAUCAAGGAAGAC
AUGAAUGUCAAGUUUUUCAAUAGCAACAAAAAGAAACGAGAUGACUUCGA
AAAGCUGACUAAUUAUUCGGUAACUGACUUGAAUGUCCAACGCAAAGCAA
UACAUGAACUCAUCCAAGUGAUGGCUGAACUGUCGCCAGCAGCUAAAACA
GGGAAGCGAAAAAGGAGUCAGAUGCUGUUUCGAGGUCGAAGAGCAUCCCA
GUGACUCGAGUGUUUUGGCUGGGUUUUUCCUUGUUCGCACCGGACACCUC
CAGUGACCAGACGGCAAGGUUUUUAUCCCAGUGUAUAUUGUCGACAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAA
Decoy IL10Ra mRNA
(SEQ ID No: 4)
GGCCGGCGGGUUUCUGACAUCCGGCGGGUUUCUGACAUCCGGCGGGUUUC
UGACAUCCGGCGGGUUUCUGACAUCCGGCGGGUUUCUGACAUCCGGCGGG
UUUCUGACAUCCGGCGGGUUUCUGACAUCCGGCGGGUUUCUGACAUCCGG
CGGGUUUCUGACAUCCGGCGGGUUUCUGACAUUCACAACCAGGCCUCCAC
AACCAUGCUGCCUUGUCUGGUGGUUCUGCUGGCCGCUCUGCUGUCUCUGA
GACUGGGAUCUGAUGCCCACGGCACCGAACUGCCUUCUCCACCUUCUGUU
UGGUUCGAGGCCGAGUUCUUCCACCACAUCCUGCACUGGACCCCUAUUCC
UAACCAGAGCGAGAGCACCUGUUACGAGGUGGCCCUGCUGAGAUACGGCA
UCGAGAGCUGGAACAGCAUCAGCAACUGCAGCCAGACACUGAGCUACGAC
CUGACCGCCGUGACACUGGAUCUGUACCACAGCAACGGCUACCGGGCCAG
AGUUAGAGCCGUGGAUGGCAGCAGACACAGCAACUGGACCGUGACCAACA
CCAGAUUCAGCGUGGACGAAGUGACCCUGACAGUGGGCAGCGUGAACCUG
GAAAUCCACAACGGCUUCAUCCUGGGCAAGAUCCAGCUGCCUCGGCCUAA
GAUGGCCCCUGCCAAUGAUACCUACGAGAGCAUCUUCAGCCACUUCCGCG
AGUACGAGAUCGCCAUCAGAAAGGUGCCCGGCAACUUCACCUUCACACAC
AAGAAAGUGAAGCACGAGAACUUCAGCCUGCUGACCUCUGGCGAAGUGGG
CGAGUUCUGCGUGCAAGUGAAACCCAGCGUGGCCAGCAGAUCCAACAAAG
GCAUGUGGUCCAAAGAGGAAUGCAUCAGCCUGACCAGACAGUACUUCACC
GUGACAAACGUGAUCAUCUUCUUCGCCUUCGUGCUGCUGCUGUCUGGCGC
CCUGGCUUAUUGUCUGGCCCUGCAGCUGUACGUGUGACUCGAGUGUUUUG
GCUGGGUUUUUCCUUGUUCGCACCGGACACCUCCAGUGACCAGACGGCAA
GGUUUUUAUCCCAGUGUAUAUUGUCGACAAAAAAAAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAA

1. A method for improving the immunostimulatory characteristics of antigen-presenting cells comprising the introduction of polynucleotide (mRNA or DNA) molecules encoding a decoy IL-10 receptor alfa-subunit in said antigen-presenting cells.

2. The method of claim 1, wherein polynucleotide (mRNA or DNA) molecules encoding at least one functional immunostimulatory protein selected from the group comprising: CD70, caTLR4, CD40L and IFN-gamma are further introduced.

3. The method of claim 1, wherein polynucleotide (mRNA or DNA) molecules encoding the caTLR4 and IFN-gamma immunostimulatory proteins are further introduced.

4. The method of claim 3, wherein polynucleotide (mRNA or DNA) molecules encoding CD40L and/or CD70 immunostimulatory proteins are further introduced.

5. The method of any of the claims 1-4, wherein a contact of IL-10 with said antigen-presenting cells at least results in a stimulation of IL-12 secretion and/or a decrease of IL-10 secretion.

6. A method for preparing an immunotherapy agent comprising the steps of:
a) obtaining antigen-presenting cells;
b) ex vivo modifying said pool of antigen-presenting cells of step a) according to the method of any of the claims 1-5;
c) ex vivo modifying the pool of antigen-presenting cells from step b) such that they present target-specific antigen derived epitopes.

7. The method of claim 6, wherein the method of modification used in step c) is selected from the group of electroporation, viral transduction, lipofection or transfection of mRNA or DNA encoding the target-specific antigens.

8. The method of any of the claims 1-7, wherein the antigen-presenting cells are selected from the group consisting of Dendritic Cells (DCs) or B-cells isolated from or generated from the blood of a subject, or dendritic cell-lines or B-cell lines.

9. The method of any of the claims 1-8, wherein the target-specific antigen is selected from the list comprising: tumor, bacterial, viral and fungal antigen.

10. A composition comprising a combination of polynucleotide (mRNA or DNA) molecules encoding a decoy IL-10 receptor alfa subunit and polynucleotide (mRNA or DNA) molecules encoding at least one functional immunostimulatory protein selected from the group comprising: CD70, caTLR4, CD40L and IFN-gamma.

11. A composition comprising a combination of polynucleotide (mRNA or DNA) molecules encoding a decoy IL-10 receptor alfa subunit, caTLR4 and IFN-gamma immunostimulatory proteins.

12. The composition of claim 18, further comprising polynucleotide (mRNA or DNA) molecules encoding CD40L and/or CD70 immunostimulatory proteins; in particular further comprising mRNA or DNA molecules encoding CD40L.

13. The composition of any of the claims 15-18, further comprising pharmaceutically acceptable adjuvant(s).

14. A composition according to any of the claims 15-18 for use in the treatment of tumor presence, cancer, IL-10 related conditions, bacterial, viral or fungal infection, HIV infection or hepatitis infection.

15. Use of a composition according to any of the claims 15-19 as an immunostimulatory agent capable of at least potentiating an immune response in a patient in need thereof.


SEQUENCE LISTING (4 sequences)

SEQ ID NO: 1 - RNA, 1168 nt, Artificial Sequence
  TLR4ca mRNA
ggccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauucacaaccaggccuccacaaccauggcugccccuggcgcuagaaggccucuucuccuucugcugcuggccggacuggcucauggcgccucugcccuguuugaggacccugugcugagccugaacaucaccugucagaugaacaagaccaucaucggcguguccgugcugagcgugcugguggugucugugguggcugugcugguguacaaguucuacuuccaccugaugcugcuggcuggcugcauuaaguacggcaggggcgagaacaucuacgacgccuucgugaucuacagcagccaggacgaggacugggugcgcaacgagcucgugaagaaccuggaagagggcgugcccccauuccagcugugccugcacuaccgggacuucauccccggcguggccauugccgccaacaucauccacgagggcuuccacaagagccggaaagugaucgugguggugucccagcacuucauccagagccgguggugcaucuucgaguacgagaucgcccagaccuggcaguuccugagcagcagagccggcaucaucuucaucgugcugcagaagguggaaaagacccugcugagacaacagguggaacuguaccggcugcugagcagaaacaccuaccuggaaugggaggacuccgugcugggcagacacaucuucuggcggagacugcggaaggcccugcuggauggcaagagcuggaaucccgagggcacagugggcaccggcugcaauuggcaggaagccaccagcaucugauaacucgaguguuuuggcuggguuuuuccuuguucgcaccggacaccuccagugaccagacggcaagguuuuuaucccaguguauauugucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 1204 nt, Artificial Sequence
  CD40L mRNA
ggccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauucacaaccaggccuccacaaccauggucgagacauacaaccagaccagccccagaagcgccgccacaggccugccuaucagcaugaagaucuuuauguaucugcugaccguguuccugaucacccagaugaucggcagcgcccuguucgccguguaucugcacagacggcuggacaagaucgaggacgagcggaaucugcacgaggacuucguguucaugaagaccauccagcggugcaacaccggcgagagaagccugagccugcugaacugcgaggaaaucaagagccaguucgagggcuucgugaaggacaucaugcugaacaaagaggaaacuaagaaagaaaacagcuucgagaugcagaagggcgaccagaacccccagauugccgcccacgugaucagcgaggccagcagcaagaccaccuccgugcugcagugggccgagaagggcuacuacaccaugagcaacaaccucgugacccuggaaaacggcaagcagcugacagugaagcggcagggccuguacuacaucuacgcccaagugaccuucugcagcaacagagaggccagcucccaggcccccuuuaucgccagccugugccugaagucccccggcagauucgagcggauccugcugagagccgccaacacacacagcagcgccaagccuuguggccagcagucuauccaccugggcggcguguucgaacugcagccuggcgccuccguguucgugaacgugaccgauccuagccaggugucccacggcaccggcuucacaagcuucggacugcugaagcugugaugacucgaguguuuuggcuggguuuuuccuuguucgcaccggacaccuccagugaccagacggcaagguuuuuaucccaguguauauugucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - RNA, 919 nt, Artificial Sequence
  IFN gamma mRNA
ggccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauucacaaccaggccuccacaacaugaaauauacaaguuauaucuuggcuuuucagcucugcaucguuuuggguucucuuggcuguuacugccaggacccauauguaaaagaagcagaaaaccuuaagaaauauuuuaaugcaggucauucagauguagcggauaauggaacucuuuucuuaggcauuuugaagaauuggaaagaggagagugacagaaaaauaaugcagagccaaauugucuccuuuuacuucaaacuuuuuaaaaacuuuaaagaugaccagagcauccaaaagaguguggagaccaucaaggaagacaugaaugucaaguuuuucaauagcaacaaaaagaaacgagaugacuucgaaaagcugacuaauuauucgguaacugacuugaauguccaacgcaaagcaauacaugaacucauccaagugauggcugaacugucgccagcagcuaaaacagggaagcgaaaaaggagucagaugcuguuucgaggucgaagagcaucccagugacucgaguguuuuggcuggguuuuuccuuguucgcaccggacaccuccagugaccagacggcaagguuuuuaucccaguguauauugucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - RNA, 1186 nt, Artificial Sequence
  Decoy IL10Ralpha mRNA
ggccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauccggcggguuucugacauucacaaccaggccuccacaaccaugcugccuugucuggugguucugcuggccgcucugcugucucugagacugggaucugaugcccacggcaccgaacugccuucuccaccuucuguuugguucgaggccgaguucuuccaccacauccugcacuggaccccuauuccuaaccagagcgagagcaccuguuacgagguggcccugcugagauacggcaucgagagcuggaacagcaucagcaacugcagccagacacugagcuacgaccugaccgccgugacacuggaucuguaccacagcaacggcuaccgggccagaguuagagccguggauggcagcagacacagcaacuggaccgugaccaacaccagauucagcguggacgaagugacccugacagugggcagcgugaaccuggaaauccacaacggcuucauccugggcaagauccagcugccucggccuaagauggccccugccaaugauaccuacgagagcaucuucagccacuuccgcgaguacgagaucgccaucagaaaggugcccggcaacuucaccuucacacacaagaaagugaagcacgagaacuucagccugcugaccucuggcgaagugggcgaguucugcgugcaagugaaacccagcguggccagcagauccaacaaaggcaugugguccaaagaggaaugcaucagccugaccagacaguacuucaccgugacaaacgugaucaucuucuucgccuucgugcugcugcugucuggcgcccuggcuuauugucuggcccugcagcuguacgugugacucgaguguuuuggcuggguuuuuccuuguucgcaccggacaccuccagugaccagacggcaagguuuuuaucccaguguauauugucgacaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa